(19) United States
(12) Patent Application Publication
(10) Pub. No.: US 2009/0098217 A1
(43) Pub. Date: Apr. 16, 2009

(54) COMPOSITIONS AND METHODS FOR PROMOTING THICKER HAIR

(76) Inventor: Robert Blaszkowski, Maple Grove, MN (US)

Correspondence Address: CYR & ASSOCIATES, P.A., 605 U.S. Highway 169, Suite 300, Plymouth, MN, 55441, US

(21) Appl. No.: 12/244,925

(22) Filed: Oct. 3, 2008

Related U.S. Application Data
(60) Provisional application No. 60/998,363, filed on Oct. 10, 2007.

(51) Int. Cl. A61K 36/23; A61K 33/30

(52) U.S. Cl. 424/643; 424/776

(57) ABSTRACT

The invention provides compositions for thickening hair comprising a celery product and optionally zinc. The invention also provides a method of thickening hair by administering to a person a composition containing a celery product, such as celery seed, celery powder, or celery extract.

### CROSS-REFERENCE TO RELATED APPLICATIONS

This application claims the benefit of U.S. Provisional application No. 60/998,363, filed on Oct. 10, 2007, which is incorporated herein by reference in its entirety.

### FIELD OF THE INVENTION

The present invention relates to compositions and methods for stimulating growth of thicker hair and preventing or reversing hair loss.

### BACKGROUND

Both men and women can suffer from hair loss or thin hair. The most common form of hair loss is male-pattern baldness. Women also suffer from hair loss, which occurs in a more dispersed pattern and often occurs following menopause.

A number of compounds have been developed to promote hair growth, prevent hair loss or to thicken hair, in both the pharmaceutical and cosmetic fields. In the pharmaceutical arena, the US Food & Drug Administration has approved several drugs it considers safe and effective. One of these, minoxidil, marketed as Rogaine®, results in hair growth when applied to the scalp and slows the rate of hair loss in some individuals by stimulating hair follicles. Another, finasteride, marketed as Propecia®, is a drug that is taken orally to treat androgenic baldness by blocking the formation of dihydrotesterone (DHT). There are, however, adverse side effects with both pharmaceuticals. Minoxidil may cause low blood pressure, an increase in heart rate, cause weight gain due to water retention, and the scalp may become inflamed. Finasteride may cause genital deformities in male infants, impotence, decreased libido, and genital deformities in male infants, in addition to hives, rash, and swelling.

Another method for treatment of hair loss is hair transplantation. This method typically comprises transplanting the natural hair in the scalp area where hair grows to the bald area. However, the hair transplants may fall out after 2-4 weeks. Although most of the hair transplants re-grow after 3-4 months, additional transplantation surgery can be required. Thus, this transplantation method is expensive, painful and not always successful.

### SUMMARY

The present invention addresses the need for a natural, effective treatment for hair loss and thin or fine hair, which is relatively inexpensive, safe and free of adverse side effects.

The invention involves the surprising discovery that providing persons suffering from thin or thinning hair with a supplement containing celery powder and optionally zinc resulted in hair that was noticeably thicker and healthier and decreased or arrested hair loss for the subjects.

Accordingly, the invention provides a composition for thickening hair comprising a celery (Apium gravelens) product selected from celery seed, ground celery seed, celery powder, celery extract, celery oil and combinations thereof in amounts effective to promote hair thickening. In some embodiments, the hair thickening composition also comprises zinc. In some embodiments, the composition is an oral dosage composition, and the oral dosage composition may be in the form of one or more capsules or tablets. In further embodiments, the composition further comprises one or more vitamins and/or minerals from a source other than the celery product, wherein a unit oral dosage of the composition comprises at least 50% of the U.S. Recommended Daily Allowances of the vitamins and/or minerals, including, for example, magnesium, vitamin D3 and/or vitamin E. In yet other embodiments, the composition further comprises β-sitosterol, biotin, rosemary extract, and/or medium chain triglycerides.

In other embodiments, the composition comprises a topical hair thickening composition comprising a celery product. The celery product may be celery seed, ground celery seed, celery powder, celery extract, celery oil and combinations thereof in amounts effective to promote hair thickening.

The invention also provides methods for thickening hair by administering to a human or animal an effective amount of a hair-thickening composition comprising a celery product selected from celery seed, celery seed powder ground celery seed, celery powder, celery extract, celery oil and combinations thereof. In some embodiments, the composition is orally administered and in others, it is administered topically. In other embodiments, the hair-thickening composition also includes zinc. In yet other embodiments, the hair-thickening composition also includes one or more vitamins and/or minerals at 100% of their U.S. recommended daily allowance in a per-day dose of the composition.

In some embodiments, the composition is administered to the person or animal once, twice, three times or more per day. In other embodiments, the composition is administered daily for at least one month.

### DETAILED DESCRIPTION

### Definitions

The term “thickening hair” as used herein refers to any apparent increase in the thickness of hair. This includes an increase in the diameter of hair follicles, an increase in the number of hair follicles, and/or an increase in the body and strength of hair follicles. Accordingly, the term “thicker hair” as used herein, refers to hair that has increased thickness, including an increase in hair follicle diameter, an increase in the number of hair follicles and/or and increase in the strength and body of hair follicles.

As used herein, the term “composition” encompasses a product comprising the specified ingredients, as well as any product that results, directly or indirectly, from combination of the specified ingredients.

As used herein, the term “effective amount” is defined as the amount required to confer a therapeutic effect on the treated subject, and is typically determined based on age, surface area, weight and condition of the patient. A therapeutic effect for the present invention is thicker hair.

As used herein, the term, “subject” refers to a human or an animal.

### Description

The inventor discovered compositions and formulations for promoting thicker hair and methods of thickening hair. Compositions and formulations, which may be oral or topical, and methods of using the compositions for promoting thicker hair are described below.

### Compositions and Formulations

The compositions and formulations of the invention comprise a number of components described below. Dosages and formulations are also described.

Components

The compositions and formulations of the invention comprise celery extract or powder. The formulation may also comprise one or more of zinc, copper, β-sitosterol, rosemary extract, biotin, vitamin D, vitamin E, magnesium, saw palmetto, arjuna (Terminalia arjuna), inositol, ginkgo biloba, flavone glycosides, other vitamins, minerals, herbal extracts, oils and lipids, such as triglycerides.

The celery extract or powder may comprise ground celery, ground or whole celery seeds, ground celery leaves, ground celery roots, ground celery stalks, ground celery hearts, ground whole celery, celery oil, celery oleoresin, celery juice or combinations thereof. The celery extract may be a powder, oil, oil extract, an alcohol or other solvent extract or an aqueous extract.

In some embodiments, the extract or powder is prepared from celery of the species Apium graveolens. In other embodiments, the celery is from other edible Apium species. Other types of edible celery, including mountain celery, or Cryptotaenia japonica may also be used in the formulations.

Celery seed powder is made by milling or grinding whole seeds. Celery seed oil is produced by steam distillation. Celery oleoresin is typically made by extracting crashed dried celery seeds with organic solvents like food grade ethyl acetate, filtering, and then desolvating under vacuum. Methods for making celery products are described in Malhorta, S K, Celery, chapter 18, pages 317-336, in Handbook of Herbs and Spices, Volume 3, K. V. Peter editor, 2006, CRC Press, Boca Raton, Fla., and references cited therein.

The amount of celery extract or powder can vary from about 10 mg to 2000 mg or more in a daily or twice daily dose.

Without wishing to be bound by theory, celery products may be acting to promote hair thickening by improving blood circulation. Aqueous celery extract in the diet has been shown to lower total cholesterol, low density lipoprotein cholesterol, and triglygeride concentrations in the blood of rats. [Tsi, et al., Effects of aqueous celery (Apium graveolens) extract on lipid parameters of rats fed a high fat diet, Planta Med. 1995; 61 (1): 18-21; Tsi and Tan, The mechanism underlying the hypocholesteroiaeraic activity of aqueous celery extract, its butanol and aqueous fractions in genetically hyperchoiesteroiaemic RICO rats. Life Sci, 2000. 66(8):755-67.] Butanol celery extract has also been shown to lower total cholesterol. [Tsi and Tan, Life Sci. 2000, 66:755]. The inventor has found that the celery composition lowers his blood pressure and other users of the composition have also observed a lowering of their blood pressure. Thus, another benefit of consumption of the celery products of the invention is the lowering of blood pressure.

Phthalides are thought by some to be one of the active ingredients in celery, accounting for its biological effects. Phthalides have the structure I, where optionally the benzene ring of structure I is dihdydro or hexahydro, i.e., has only two double bond or no double bonds, and where the ring structure is substituted at one or two positions.

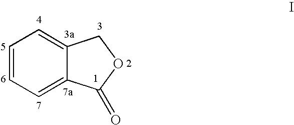

An exemplary phthalide that is found in celery is 3-n-butylphthalide (structure II below).

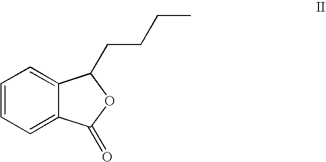

[Beck J J, Chou S-C, The structural diversity of phthalides from the Apiaceae. J. Nat, Prod. 2007, 70:891-900.]

Phthalides can be used as an indicator to quantify the bioactivity of a celery product for use in the invention. In some embodiments of the invention, the daily dosage of the hair-thickening composition has at least as much celery phthalides as 200 mg of celery seed powder. In other embodiments, the per day dosage of the hair-thickening composition has at least as much celery phthalides or celery extract, celery powder or ground celery seed as 250 mg, 300 mg, 400 mg, 500 mg, 700 mg, 800 mg, 1000 mg or 1500 mg of celery seed powder.

Phthalides can be extracted, quantified, separated and identified as described in Uhlig, J W et al., 1987, J. Food Sci. 52:658-660. In some embodiments, the celery extract or powder is standardized to contain 85% of 3-n-butylphthaiide, along with other celery pthalides.

In other embodiments, particularly those using compositions containing aqueous extracts of celery, the celery extract may have lower levels of phthalides or even no phthalides. It is not clear that phthalides are the bioactive component of celery in the invention, since Tsi et al. reported that aqueous celery extract containing very low phthalide levels was effective in lowering rat cholesterol. [Tsi D, Das N P, Tan B K. Effects of aqueous celery (Apium graveolens) extract on lipid parameters of rats fed a high fat diet, Planta Med. 1995 February;61(1):18-21; Tsi D, Tan B K, The mechanism underlying the hypocholesterolaemic activity of aqueous celery extract, its butanol and aqueous fractions in genetically hypercholesterolaernic RICO rats, Life Sci, 2000 Jan. 14;66(8):755-67.]

In some embodiments, the hair-thickening composition or formulation includes zinc. The zinc may be from a source other than the celery product. In another embodiment, the hair-thickening oral dosage composition comprises at least 30 mg zinc in a per-day dose. In yet another embodiment, the hair-thickening oral dosage composition comprises at least 4 mg zinc in a per-day dose. The amount of zinc in a daily dose or twice daily dose may range from about 1 or 2 mg to about 60 mg or more. In some embodiments, the composition comprises at least 2 mg of zinc.

Zinc is optional in the compositions in part because the amount of zinc found to be effective is close to that ordinarily consumed in a well-balanced diet. The recommended daily allowance (RDA) of zinc is 15 mg, so in some embodiments, the methods herein involving administering 15 or 30 mg zinc per day are administering only 100-200% of the RDA.

Zinc may be isolated from a natural food or other source. Zinc may be in the form of a Zinc salt, including without limitation, zinc picolinate, zinc gluconate, zinc citrate, zinc arginate, zinc aspartate and combinations thereof.

Zinc may be acting by inhibiting 5-alpha-dihydrotestosterone (DHT) formation. High levels of DHT are known to be linked to male pattern baldness. DHT is formed by the enzyme 5-alpha-reductase. [Jankovic S M, Jankovic S V, 1998, Dermatology Online Journal 4(1):2.] Zinc has effects on the conversion of testosterone to DHT. In vitro, low concentrations of zinc were shown to enhance conversion of testosterone to DHT, while mM concentrations inhibited it. [Hudson R W, Comparison of nuclear 5 alpha-reductase activities in the stromal and epithelial fractions of human prostatic, tissue, J. Steroid Biochem. 1987 March;26(3):349-53.] Another study found that injecting 5 or 10 mg of zinc in rat prostate significantly reduced 5-alpha-reductase activity. [Fahim M S, Wang M, Sutcu M F, Fahim Z. Zinc arginine, a 5 alpha-reductase inhibitor, reduces rat ventral prostate weight and DNA without affecting testicular function. Andrologia. 1993 November-December;25(6):369-75.]

The composition or formulation may also include one or more phytosterols, including β-sitosterol. Phytosterols are isolated from plant sources. The amount of phytosterols, including β-sitosterol in a daily dose or twice daily dose may range from about 50 μg to about 50 mg.

The composition or formulation may also include herbal extracts, including rosemary extract, which may be in the form of rosemary leaf powder or extract. Amounts of rosemary leaf powder may range from about 5 mg to about 70 mg in a daily or twice daily dose. Other herbal extracts that may be included in the formulation are, without limitation, saw palmetto, arjuna (Terminalia arjuna), ginkgo biloba, green tea extract, and combinations thereof. The arjuna is typically a powder of arjuna bark or an extract thereof. Ginkgo bilboa is typically a product of ginkgo bilboa leaves. The amount of arjuna in a composition may include at least 75 mg of arjuna bark powder or a bioequivalent amount of arjuna extract. A composition containing saw palmetto may, in one embodiment, include at least 400 mg in a daily dose. A composition containing ginkgo biloba may contain in a daily dose at least 7.5 mg ginkgo biloba extract standardized to contain at least 24% flavone glycosides.

Other herbal extracts and/or oils may be included in the formulations or compositions, including, without limitation, nut oils, borage oil, flax seed oil and evening primrose oil.

The composition or formulation may also include vitamins such as, without limitation, vitamin D, vitamin D3, vitamin E, B-vitamins, such as vitamin B6 and vitamin B12, biotin, inositol, lysine and L-lysine. The amount of biotin in a daily dose may range from about 100 ug to about 100 mg. The amounts of vitamin D3 and vitamin E in a daily dose may range from about 20 IU to about 400 IU, and from about 1 IU to about 20 IU, respectively. The amount of inositol in a daily dose may be at least 35 mg. The amounts of vitamin B6 and vitamin B12 in a daily dose may be at least 1 mg and 3 μg, respectively.

The composition or formulation may also include other minerals, such as, without limitation, copper and magnesium. The minerals are generally in the form of salts, such as, without limitation, cupric sulfate and magnesium chloride. The amount of copper in a formulation may be at least 1 mg per daily dose.

The composition or formulation may also include lipids, such as, without limitation, triglycerides, including medium chain triglycerides (MCT) and magnesium stearate. A formulation may include from about 1 mg to about 50 mg of MCT and/or magnesium stearate in a daily dose.

The zinc and other optional components of the compositions other than a celery product may be from a source other than the celery product. In some embodiments, the zinc, vitamins, minerals and other components are purified, i.e., synthesized or separated from a natural food.

Some of the above components may act to block DHT. These include β-sitosterol, zinc, biotin and rosemary, and saw palmetto, among others. Some of these, including β-sitosterol may be much more effective (such as over 1000 times more effective) than a herbal extract such as saw palmetto in blocking DHT.

The composition or formulation may also include other components for promoting thicker hair or hair growth, including, without limitation, minoxidil, finasteride or other DHT inhibitors.

Compositions

In particular embodiments, the hair-thickening compositions for oral administration comprise a celery product, zinc from a source other than the celery product, and/or one or more vitamins and/or minerals from a source other than the celery product. In some embodiments, where the composition comprises one or more vitamins and/or minerals, a one day dose of the composition comprises a plurality of the vitamins and/or minerals at 100 to 200% of their U.S. Recommended Daily Allowances.

One particular embodiment of the hair-thickening composition comprises a celery product, zinc, saw palmetto, and a plurality of vitamins and/or minerals.

Another embodiment of the composition contains a celery product, zinc, saw palmetto, biotin, vitamins B6 and B12, and copper.

Yet another embodiment of the hair-thickening composition comprises a celery product, zinc pincolinate, Ginkgo biloba, iron and a plurality of vitamins and minerals.

An additional embodiment of the hair-thickening composition comprises a celery product, zinc pincolinate, Ginkgo biloba, and a plurality of vitamins and minerals.

In one embodiment, the per day dosage of the composition for oral administration includes at least 400 mg celery seed or celery seed powder and at least 15 mg zinc, or at least 700 mg celery seed or celery seed powder and at least 15 mg zinc.

In other embodiments, the per day dosage of the composition for oral administration includes at least 300 mg celery seed or celery seed powder and at least 2 mg zinc, or at least 700 mg celery seed or celery seed powder and at least 4 mg zinc.

In one embodiment of the oral dosage composition a unit oral dosage of the composition comprises a celery product containing at least as much celery phthalides as 200 mg celery seed or celery seed powder, at least 1 mg of zinc, at least 200 mg saw palmetto, at least 150 micrograms biotin, at least 1 mg vitamin B6, at least 3 micrograms vitamin B12, and at least 1 mg copper. In one embodiment of the oral dosage composition the composition contains a celery product, zinc, arjuna (Terminalia arjuna) or arjuna extract, biotin, inositol, and ginkgo bilboa. The arjuna is typically a powder of arjuna bark or an extract thereof. Ginkgo bilboa is typically a product of ginkgo bilboa leaves. In a particular embodiment, a daily dose of the composition comprises a celery product containing at least as much celery phthalides as 200 mg celery seed or celery seed powder, at least 1 mg zinc, at least 150 mg arjuna bark powder (or a bioequivalent amount of arjuna extract), at least 70 mg inositol, at least 300 micrograms biotin, and ginkgo bilboa extract containing at least 15 mg flavone glycosides (e.g., at least about 60 mg gingko bilboa extract standardized to contain at least 24% flavone glycosides).

In another embodiment, a daily dose of the oral dosage composition comprises a celery product containing at least as much celery phthalides as 750 mg celery seed powder; at least 4 mg zinc, at least 200 mg arjuna bark powder (or a bioequivalent amount of arjuna extract), at least 80 mg inositol, at least 800 micrograms biotin, and ginkgo bilboa extract containing at least 15 mg flavone glycosides.

In particular embodiments a single dose of the oral dosage composition comprises a celery product containing at least as much celery phthalides as 3 00 mg celery seed or celery seed powder, at least 1 mg zinc, at least 75 mg arjuna bark powder, at least 35 mg inositol, at least 350 micrograms biotin, and ginkgo bilboa extract containing at least 7.5 mg flavone glycosides.

In particular embodiments of the oral dosage composition for promoting hair thickening, a unit oral dosage of the composition comprises at least as much celery phthalides as 200 mg of celery seed powder or 400 mg of celery seed powder. In other embodiments, the unit oral dosage further includes at least 7.5 mg zinc or at least 2 mg zinc. The term “unit oral dosage” as used herein refers to a dosage intended to be consumed at one time.

In other embodiments of the oral dosage composition for promoting hair thickening, a unit oral dosage of the composition comprises about 15 IU of vitamin E, about 400 IU of vitamin D3, about 300 μg of biotin about 4-5 mg of zinc from zinc picolinate and about 1100 to about 1200 mg of a blend of ground celery, β-sitosterol and rosemary leaf powder. In another embodiment, the oral dosage composition comprises about 560 mg of ground celery seed, about 8 mg of β-sitosterol, about 7 mg of rosemary leaf powder extract, about 12 mg of zinc picolinate (containing about 20% zinc), about 10 mg of biotin, about 200 IU of vitamin D3, about 7-8 IU of vitamin E, about 8 mg of magnesium stearate and about 9 mg of medium chain triglycerides.

Topical Compositions

Another embodiment of the invention provides a topically applied hair-thickening composition comprising celery extract.

In some embodiments, the topically applied hair-thickening composition comprises zinc, e.g., as zinc gluconate or zinc picolinate.

In some embodiments, the topically applied hair thickening composition comprises one or more vitamins and/or minerals. The per-day dose of the composition may contain one or more vitamins and/or minerals in amounts of at least 100% of the U.S. RDA.

The topically applied hair-thickening composition may be an oil, lotion, ointment, cream, emulsion, or aqueous solution. In some embodiments, it is a shampoo or hair conditioner.

Formulations

The components of the compositions and formulations of the invention may vary, but all include celery extract, powder or oil. The compositions described herein may be prepared in any suitable form appropriate for the desired use. Examples of suitable dosage forms include oral, parenteral or topical dosage forms.

In some embodiments of the orally administered hair-thickening compositions, the composition is in the form of one or more capsules or tablets. In other embodiments, the composition is in the form of a liquid. The liquid may be an oil-based or aqueous fluid. Suitable dosage forms for oral use include tablets, dispersible powders, granules, capsules, soft-gel capsules, suspensions, syrups, and elixirs. Inert diluents and earners for tablets include, for example, calcium carbonate, sodium carbonate, lactose, and talc. Tablets may also contain granulating and disintegrating agents, such as starch and alginic acid; binding agents, such as starch, gelatin, and acacia; and lubricating agents, such as magnesium stearate, stearic acid, and talc. Tablets may be uncoated or may be coated by known techniques to delay disintegration and absorption. Inert diluents and earners which may be used in capsules include, for example, calcium carbonate, calcium phosphate, and kaolin. Capsules may be gelatin or similar capsules. Suspensions, syrups, and elixirs may contain conventional excipients, for example, methyl cellulose, tragacanth, sodium alginate; wetting agents, such as lecithin and polyoxyethylene stearate; and preservatives, such as ethyl-p-hydroxybenzoate. Dosage forms for oral administration can also be formulated as food preparations using materials which are conventionally used in the food processing industry, such as proteins, sugars and other carbohydrates, extenders, fillers, preservatives, and the like. Examples of other compounds for use in formulating capsules, tablets and liquid preparations are described in detail in the U.S. Pharmacopeia. Tablet preparation and capsule filling is well known to skilled artisan and is described, for example, by Edward Rudnic and Joseph B. Schwartz, Oral Solid Dosage Forms, in Volume II, Remington: The Science and Practice of Pharmacy, Chpt. 92, 1615, 1615-1637 (Alfonso R. Gennaro, ed., 19^{th} ed. 1995), incorporated herein by reference.

The oral solid dosage form used in the present invention can be a chewable food item that includes the hair thickening composition. In addition to the hair thickening composition, the chewable food item can contain one or more nutrients such as soy protein isolate, soy protein hydrolysate, calcium caseinate, whey protein isolate, whey protein concentrate, milk protein isolate, skim milk powder, yogurt solids, or hydrolyzed bovine gelatin in combination with oils, binders, fillers, and processing aids known in the art. The chewable food item may be formed of a mass having the desired ingredients into a shape, preferably a bar having a circular, semicircular, or rectangular cross-section by, for example, extrusion, and cut into chewable food item dosage forms of about 50 to about 200 grams each. In some embodiments, such dosage form includes a blood sugar and glycosylation maintaining effective amount of the hair thickening composition.

Oral liquid dosage forms may be prepared with one or more vehicles and include solutions, emulsions, and suspensions. A vehicle can be any potable substance or mixture of potable substances that are liquid at room temperature and that do not interfere with the effectiveness of the hair thickening composition. In some embodiments, water, ethanol, and oils, including vegetable oils and seed oils, are used. Emulsions and suspensions can also be used.

Preparation of oral liquid dosage forms is well known to the skilled artisan and is described, for example, by J. G. Narin, Solutions, Emulsions, Suspensions and Extracts, in Volume II Remington: the Science and Practice of Pharmacy, Chpt. 86, 1495, 1495-1521 (Alfonso R. Gennaro, ed., 19^{th} ed., 1995, incorporated herein by reference.

These oral liquid dosage forms can be ready-made, that is introduced into commerce in the liquid form in which they are to be administered. In some embodiments, oral liquid dosage forms of the ready-made type are in the form of a beverage that can be carbonated. Alternatively, a dry concentrate of the oral liquid dosage form can be supplied in the form of a powder that can be mixed by the practitioner or consumer with a potable liquid (e.g. milk, water, fruit juice) to form the oral liquid dosage form prior to administration. Powders can contain additives known in the art to prevent caking of the powder and maintain desirable free-flowing characteristics of the powder.

Dosage forms suitable for parenteral administration include solutions, suspensions, dispersions, emulsions, and the like. They may also be manufactured in the form of sterile solid compositions which can be dissolved or suspended in sterile injectable medium immediately before use. They may contain suspending or dispersing agents known in the art. Examples of parenteral administration are intraventricular, intracerebral, intramuscular, intravenous, intraperitoneal, rectal, and subcutaneous administration.

Suitable topical dosage forms include gels, creams, lotions, ointments, powders, aerosols and other conventional forms suitable for direct application of medicaments to skin or mucous membranes. Topical ointments, pastes, creams, and gels can include, in addition to the active ingredients, customary excipients, for example animal and vegetable fats, waxes, paraffins, starch, tragacanth, cellulose derivatives, polyethylene glycols, silicones, bentonites, silicic acid, talc, and zinc oxide, or mixtures of these substances. Topical powders and sprays can include, in addition to the active ingredients, the customary excipients, for example lactose, talc, silicic acid, aluminium hydroxide, calcium silicate and polyamide powder, or mixtures of these substances. Sprays can additionally contain the conventional propellants.

### Methods of Thickening Hair

Methods of thickening hair comprise administering to a subject an effective amount of a hair-thickening composition comprising a celery product. The composition can be administered orally, parenterally or topically.

In one embodiment, the method of thickening hair includes orally administering to a person an effective amount of a hair-thickening composition comprising a celery product. The composition may comprise at least 200 mg celery seed or celery seed powder and at least 1 mg zinc, at least 400 mg celery seed or celery seed powder and at least 2 mg of zinc. Another embodiment comprises administering a composition comprising at least 200 mg celery seed or celery seed powder and at least 1 or 2 mg zinc twice per day.

In particular embodiments, the method of thickening hair includes orally administering the composition comprising the hair-thickening composition to a subject daily for at least one month.

In other embodiments, the method of thickening hair comprises orally administering one or more of the compositions described above to a subject once, twice, three or more times daily. In some embodiments, the duration of daily administration can be from 7 days to 50 years or more.

In other embodiments the method of thickening hair comprises administering a topical formulation containing a celery product to the skin or scalp of a subject.

In yet other embodiments, the method of thickening hair comprises administering an oral or topical composition containing a celery product and optionally zinc to a person one or more times per day, along with the use of an apparatus for stimulating hair growth, such as, without limitation, those described in U.S. Pat. Nos. 6,497,719; 7,194,316, 7,258,695 and similar devices.

### Kits

The compositions described herein may be included in a kit. The kit may comprise a composition for thickening hair comprising a celery product and optionally zinc. The composition may be an oral, parenteral or topical formulation. The kit may also include an apparatus for thickening hair, such as, without limitation, those described in U.S. Pat. Nos. 6,497,719; 7,194,316, 7,258,695 and similar devices. The kit may also include instructions for use.

The invention will be further described in the following examples, which do not limit the scope of the invention described in the claims.

### EXAMPLES

At least twelve subjects, both male and female have stated that taking an oral formulation containing about 500 mg of celery powder or extract either once or twice or three times per day resulted in thicker hair after about one month. In several cases, the subjects' hair care professionals have stated that the subjects' hair is thicker after taking the oral composition. The subjects have continued to take the oral formulation for up to nine months and longer.

### Example 1

One male subject observed that when he consumed one spoonful of celery seed powder orally each week, his hair appeared to become thicker and have more body. Somewhat greater hair thickening was observed when the subject consumed two rounded teaspoons of celery seed powder per week. He then consumed 1000 mg celery seed powder and 30 mg of zinc in the form of zinc gluconate, divided into two doses each day, as daily supplementation, and observed greater hair thickening than with celery powder alone.

### Example 2

Another subject was a 43-year old male. He took 500 mg celery seed powder in one dose daily for 4 weeks and noticed that his hair seemed thicker. He then took a composition containing 500 mg celery seed powder and 15 mg zinc twice daily for 4 more weeks. He observed that his hair was definitely thicker and stood higher on his head.

### Example 3

Subject 3 was a 50 year old male with soft limp thinning hair. After 6 weeks of taking a composition containing 500 mg celery seed powder and 15 mg zinc in zinc gluconate by oral administration twice daily he went to his hair care professional, who noted that his hair was much thicker.

### Example 4

Subject 4 was a woman in her 40s. After taking the oral composition in one dose each day, each containing 500 mg celery seed powder and 15 mg zinc, for 4 weeks, she reported that her hair was thicker and more manageable. She has continued to take the oral composition for four months and more and continues to observe thicker and more manageable hair.

### Example 5

Subject 5 was a man in his 40s experiencing hair loss. After taking the oral composition in two doses each day, each dose containing 500 mg celery seed powder and 15 mg zinc, for two weeks he reported that he definitely was finding fewer hairs in his shower drain. He continued to take the oral composition for four months and more and has noticed that his hair continues to be thicker.

### Example 6

Subject 6 was a female in her late 30's with thin, fine hair. After taking the oral composition in two doses each day, each dose containing 500 mg celery seed powder, for one month she noticed her hair was thicker. She then added a multi-vitamin to her daily consumption, and felt that this increased the thickening of her hair. After 4 weeks of consuming both the celery seed powder and the vitamin tablet, she went to her hair care professional, who cut and foiled her hair. Her hair care professional commented that the hair was thicker, healthier and stronger. She continues to take the oral composition for seven months and more and continues to have thicker hair.

### Example 7

Some individuals found that their cholesterol levels dropped after they took an oral composition containing 500 mg celery seed powder and 15 mg zinc (for how long?). The individuals were both male and female and ranged in age from 40 to 51 years. Those with initial cholesterol levels of about 200 found that their cholesterol levels were lowered from about 15 to 20%. One individual with an initial cholesterol level of about 175 found his cholesterol lowered about 4% after taking the oral composition.

Other Embodiments

It is to be understood that while the invention has been described in conjunction with the detailed description thereof, the foregoing description is intended to illustrate and not limit the scope of the invention, which is defined by the scope of the appended claims. Other aspects, advantages and modifications are within the scope of the following claims.

All publications, patent publications and applications mentioned in this specification are herein incorporated by reference in their entireties to the same extent as if each individual publication or patent application was specifically and individually indicated to be incorporated by reference.

What is claimed is:

1. A composition for promoting hair thickening comprising (a) a celery product selected from the group consisting of celery seed, celery seed powder, ground celery seed, celery powder, celery oil, celery extract and combinations thereof and (b) zinc, in amounts effective to promote hair thickening.

2. The composition of claim 1, wherein the composition is an oral dosage composition.

3. The composition of claim 2, wherein the composition is in the form of one or more capsules or tablets.

4. The composition of claim 2, wherein a unit oral dosage of the composition comprises at least 300 mg of celery seed or celery seed powder and at least 2 mg of zinc.

5. The composition of claim 2, wherein a unit oral dosage of the composition comprises at least as much celery phthalides as 300 mg of celery seed powder and at least 2 mg of zinc.

6. The composition of claim 2, further comprising one or more vitamins and/or minerals from a source other than the celery product, wherein a unit oral dosage of the composition comprises at least 50% of the U.S. Recommended Daily Allowances of the vitamins and/or minerals.

7. The composition of claim 2, further comprising one or more of β-sitosterol, biotin, rosemary extract, and medium chain triglycerides.

8. The composition of claim 7 further comprising vitamin D3, vitamin E or both.

9. A topical hair thickening composition comprising celery extract.

10. A method of thickening hair comprising administering to a subject an effective amount of a hair-thickening composition comprising a celery product selected from celery seed, celery seed powder ground celery seed, celery powder, celery extract, celery oil and combinations thereof.

11. The method of claim 10, wherein the hair-thickening composition is administered topically to a subject's scalp or skin.

12. The method of claim 10, wherein the hair-thickening composition is administered orally.

13. The method of claim 12, wherein the hair-thickening composition further comprises zinc.

14. The method of claim 13, wherein the hair-thickening composition further comprises one or more vitamins and/or minerals at 100% of their U.S. recommended daily allowance in a per-day dose of the composition.

15. The method of claim 12, wherein the celery product comprises ground celery seed.

16. The method of claim 12, wherein the method comprises administering a per-day dose of the hair thickening composition comprising a celery product comprising at least as much celery phthalides as 400 mg of celery seed powder.

17. The method of claim 16, wherein the per-day dose of the hair-thickening composition further comprises at least 2 mg zinc.

18. The method of claim 16, wherein the per-day dose of the hair-thickening composition comprises at least 400 mg ground celery seed or celery seed powder and at least 2 mg zinc.

19. The method of claim 17, wherein the method comprises administering a hair-thickening composition comprising at least 200 mg ground celery seed or celery seed powder and at least 2 mg zinc twice per day.

20. The method of claim 12 comprising administering the hair-thickening composition daily for at least one month.
